(19) United States
(12) Patent Application Publication
(10) Pub. No.: US 2005/0065492 A1
(43) Pub. Date: Mar. 24, 2005

(54) Absorbent product line and device for identifying absorbent products

(76) Inventors: Robert T. Cole, Jackson, NJ (US); Prelo M. Hood, Schwenksville, PA (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 10/668,297

(22) Filed: Sep. 24, 2003

(51) Int. Cl. A61F 13/15; A61F 13/20

(52) U.S. Cl. 604/385.01

(57) ABSTRACT

A product line that is tailored to the fit and absorbency needs of users, and a visual identification device that identifies absorbent products predicted to satisfy users' fit and absorbency requirements. The identification device has product designations that identify absorbent products, size designations that relate to the sizes of the absorbent articles, and absorbency designations that relate to the absorbent properties of the absorbent articles. Each product designation is associated with a combination of one size designation and one absorbency designation so that the size and absorbency of each product is clearly identified. At least two product designations are associated with one of the size designations.

### BACKGROUND OF THE INVENTION

1. Field of the Invention

The present invention relates to an absorbent product line tailored to the fit and absorbency needs of users, and a device to be used to identify absorbent articles within an absorbent product line. Specifically, the invention relates to a visual identification device, such as one that may be displayed on a package or display of absorbent articles, that identifies one or more absorbent products that are part of a product line that encompasses a range of sizes and absorbencies. The visual identification device has product designations that represent absorbent products; each product designation is associated with a size designation and an absorbency designation. A user may use the device to select an absorbent product that is predicted to meet the user's size and absorbency needs.

2. Description of Related Art

Absorbent articles, such as bladder control products or incontinent products, are designed to absorb and contain fluid to prevent soiling of the body and clothing. These absorbent articles come in a variety of sizes within a product brand or category, to provide a range of sizes and absorbencies for typical users. In traditional product lines, the absorbency of each article is proportional to the size of the article; a shorter or smaller product has lower absorbency than a longer or larger product. A user typically selects a product based solely on the user's expected absorbency needs, and the product size is pre-determined based on that absorbency level. Thus, a person has few product options within a traditional product line, and must choose products that are not necessarily the proper size in order to obtain the desired absorbency.

It has been discovered, however, that users of these absorbent products have a demonstrated preference for certain product sizes, independent of their absorbency needs. In other words, a user may believe that a certain product size fits the user better than other sizes, and if absorbency were not a factor, the user would choose to wear a product of the size that fits the user best. For this reason, a user may be dissatisfied with a traditional product line because he or she prefers a smaller size product, but needs more absorbency than what traditional small products offer. The opposite is also true—a user may prefer a longer product, but may not need all the absorbency of a traditional long product. Thus there is a need for a product line that provides product offerings tailored to the specific fit and absorbency needs of users, such as a product line that offers products having more than one level of absorbency within each size offering.

One challenge in offering a product line that comprises multiple absorbencies for a single product size offering is to clearly identify to users which product within a product line meets both their size and absorbency requirements. With traditional product lines, in which size is proportional to absorbency, if a user finds that one product no longer meets his or her absorbency needs, then he or she simply selects the next larger (or smaller) size product. In contrast, when the product line includes products having multiple levels of absorbency for a particular size offering, identifying a product that meets both size and absorbency requirements may become difficult and confusing.

Furthermore, products within a consumer product line are typically displayed in close proximity to one another on the store shelves and often use similar packaging and/or nomenclature to designate a particular product line. Typically, each product in a product line is identified and differentiated from the other products in that product line by a differentiating means on the package, for instance, a unique name or size indicator. However, such packaging and display techniques often provide the user with an incomplete understanding of the products that are available, particularly when the product line includes products having multiple levels of absorbency for a particular size offering. As such, the user may not appreciate the relationship between the products in the product line, and may not realize that a different product may better suit the user's needs.

Thus a need exists to provide an identifying device that clearly identifies the absorbency level and size of various products in a product line to assist a user with selecting a product that meets both the user's absorbency and fit needs.

### SUMMARY OF THE INVENTION

In response to the difficulties encountered in the prior art, an absorbent product line is disclosed that offers a user products that are tailored to the fit and absorbency needs of users. In addition, a device is disclosed that clearly identifies the absorbency level and size of each absorbent product in a product line.

In various embodiments of the invention, an absorbent product line may include two or more absorbent products that are each characterized by a combination of an absorbency designation and a size designation. Each of the absorbent products is characterized by a different combination of absorbency designation and size designation. In one embodiment of the invention, at least two of the absorbent products in the product line are characterized by the same absorbency designation, and different size designations. In another embodiment of the invention, at least two of the absorbent products in the product line are characterized by the same size designation, and different absorbency designations.

In various embodiments of the invention, an absorbent product line may include a plurality of packages, and a plurality of products having a similar construction, wherein each product has a size designation and an absorbency designation. Each package contains products having the same size and absorbency characteristics, and has the same size designation and absorbency designation as the products it contains. In one embodiment, at least two packages have the same size designation and different absorbency designations. In another embodiment of the invention, at least two packages have the same absorbency designation and different size designations.

One embodiment of the invention is a visual identification device having two or more absorbent product designations that identify absorbent products, two or more size designations, and two or more absorbency designations. Each product designation is associated with a combination of one size designation and one absorbency designation. At least one of the size designations is associated with two or more product designations. The device employing this association assists a user in selecting a product that is predicted to satisfy the user's fit and absorbency requirements. In one embodiment of the invention, the product designations identify bladder control pads, but other products may be identified.

In various embodiments of the invention, the visual identification device may be, for example, a chart, a graphical representation, a table or a list. For example, in some embodiments of the invention, the device is a graphical representation in the form of a chart having a size axis and an absorbency axis, the size axis corresponding to size designations and the absorbency axis corresponding to absorbency designations. The chart has two or more absorbent product designations, each associated with a combination of one size designation and one absorbency designation.

In various embodiments of the invention, the absorbent product designations may be textual descriptions or graphical codes that identify an absorbent product. For example, the absorbent product designations may be a product name, product brand name, product line name, a product shape, words, letters, numbers, boxes, partitions, color codes, symbols, graphical points, bars, lines, or a combination thereof. In various embodiments of the invention, the size designations and the absorbency designations may be textual descriptions or graphical codes, such as, for example, words, letters, numbers, boxes, partitions, color codes, symbols, graphical points, bars, lines, or a combination thereof. In various embodiments of the invention, the device also includes a content indicator associated with one of the product designations.

In various embodiments of the invention, the visual identification device may be displayed, for example, on packaging materials, in-store display materials, advertising materials, or promotional materials. One embodiment of the invention involves a package for containing absorbent products, where the package comprises two or more absorbent product designations, two or more size designations and two or more absorbency designations. Each of the product designations is associated with a combination of one of the size designations and one of the absorbency designations, and at least one of the size designations is associated with two or more product designations. In such an embodiment, the package may also include a content indicator that identifies the absorbent product contained within the package.

Another embodiment of the invention is a method for marking an absorbent article package. The method comprises marking a package with a series of two or more absorbent product designations, two or more size designations and two or more absorbency designations. Each of the product designations identifies an absorbent product that is associated with one absorbency designation and one size designation. At least one of the size designations is associated with two or more product designations.

These and other objects, features and advantages of the invention will be apparent through the detailed description of the preferred embodiments and the drawings attached hereto. It is also to be understood that both the foregoing general description and the following detailed description are exemplary and explanatory and are not restrictive of the scope of the invention.

### BRIEF DESCRIPTION OF THE DRAWINGS

The invention may be understood more readily by reference to the accompanying drawings, in which:

FIG. 1 illustrates a device used to identify absorbent articles.

FIG. 2 illustrates an absorbent product line.

### DETAILED DESCRIPTION OF THE PREFERRED EMBODIMENTS

The present invention relates to a device for selecting, from a product line having a number of products, an absorbent product that will provide desired fit and absorbency characteristics for the intended user. The identifying device of the present invention provides an association between an identified product and a combination of size and absorbency level characteristics. With the present invention, a user can evaluate a line of similar products and select the one that best satisfies the user's needs based on the associations between each product and its size and absorbency characteristics. The user's desired size and absorbency level are typically known by the user, and absorbent article manufacturers have developed numerous techniques for predicting typical user requirements and producing a line of products that meet a broad range of anticipated requirements. As used herein, the term “user” is not intended to be exclusive, and refers to, inter alia, a person who uses or wears the absorbent article or purchases or otherwise selects such products for their own use, for the use of another person, or for resale purposes.

Throughout this description, the expressions “absorbent article” or “absorbent product” refer to articles that absorb and contain body exudates, and, more specifically, refer to articles that are placed against or in proximity to the body of the user to absorb and contain various exudates from the body. The term “disposable” as it is used herein, denotes absorbent articles that are not intended to be laundered or otherwise restored or reused as an absorbent article (i.e., they are intended to be discarded after a single use and, preferably, to be recycled, composted or otherwise disposed of in an environmentally compatible manner). Exemplary absorbent articles and absorbent garments include, but are not limited to, adult incontinent products, feminine hygiene products and bladder control products. The invention can be used with all of the foregoing classes of absorbent articles and garments or any other classes of article not specifically listed herein, without limitation, and whether disposable or otherwise.

The device of the present invention uses an association between a product, its size, and its absorbency. The associations employed by the device are preferably based on a model that predicts, based on data related to fit and absorbency, the suitability of the absorbent product to the expectations of the user. Data related to fit and absorbency of an absorbent article may be derived by a number of methods known in the art. For example, fit-related data may be derived by wear-testing various sizes of the article having a design of interest, preferably on an appropriate population of potential users. A relationship may be developed between a population of users who prefer to use a certain absorbent article size designation, and a rating of how well an article of the design fits a user. For example, a population of users who prefer to use “Regular” products may wear test a product of a certain design that may correspond to typical regular products. It may be within the scope of the test to also wear-test products having a larger or smaller size design to evaluate the user's rating of alternative designs. It may also be within the scope of the test to vary the population of wear-testers, for instance by size preference or by absorbency preference.

Wear-testing fit ratings can be determined through observation, measurement, or by obtaining feedback from the user or the user's caregiver. The rating can be based on the overall fit of the article having the design of interest, or can be broken down into ratings of fit for selected characteristics, for instance, length, width, or thickness of the article. Ultimately, this data may be used to identify a series of sizes that will fit the greatest number of users, and the products of the product line can be manufactured according to these popular sizes. Similar data can be collected for the absorbency of the products, and the products of the product line can also be made to have a range of popular absorbency levels.

In a preferred embodiment, the invention comprises a visual identification device that is inspected by a user to determine which product will best satisfy the user's size and absorbency requirements. The device has a plurality of absorbent product designations that identify absorbent products, a plurality of size designations, and a plurality of absorbency designations. Each of the product designations is visually associated with a combination of one size designation and one absorbency level designation. These associations identify the size and absorbency of each identified absorbent product.

Each product designation represents a product offering that is part of a product line. The phrases “line of products” or “product line” or “consumer product line” refer to, in general, a series of absorbent products (“products” or “product offerings”) having a common marketing approach (e.g., brand name or category) and/or technical approach. For instance, the absorbent products could all be bladder control pads or have a similar construction style (e.g., hourglass shape or panty-like configuration). The number of products and characteristics of the products within a product line depend on the product line strategy, which may be driven by many factors, including, for instance, marketing objectives, consumer needs and equipment capability. Within a product line, two or more products may share one or more characteristics, for instance, size or absorbency level or outer cover material, however each product within a product line also differs from the other products in at least one characteristic. For example, in a product line of absorbent products, some products may have the same size, but have different absorbency characteristics.

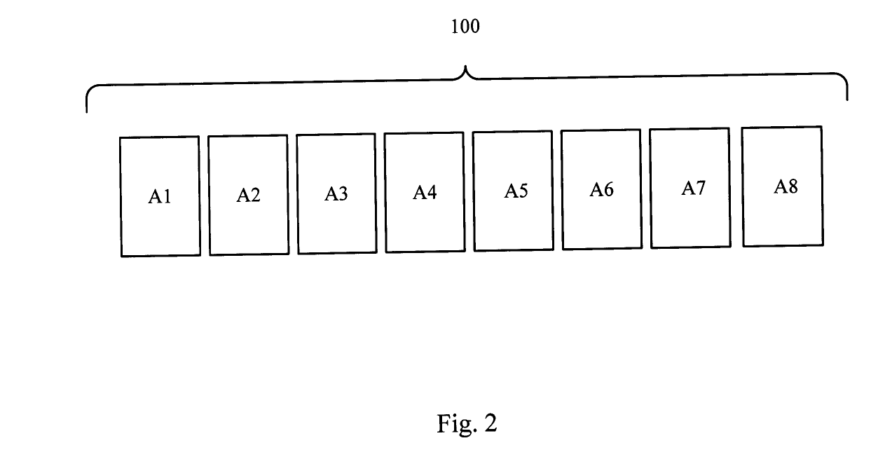

FIG. 2 illustrates an embodiment of a product line 100 of the present invention. The absorbent product line 100 of this embodiment includes a plurality of absorbent products that share a common construction, each absorbent product being characterized by a combination of size and absorbency. Products sharing the same combination of size and absorbency are contained within a package A1-A8. As used herein, the term “package” refers to a sealed package used to distribute the final absorbent product. The term “package” includes a unitary package housing absorbent products being distributed/sold to the user. The term “package” includes but is not limited to paperboard containers (e.g., boxes, cartons) or plastic containers (e.g., bags) that form the final product being distributed/sold to the user. It also may include individual packages which are connected together (e.g., by glue or shrink-wrap) to form the final product being distributed/sold to the user. As shown in FIG. 2, the product line 100 of this embodiment contains eight packages, A1-A8, however it is contemplated that the product line may include more or fewer packages of absorbent products, as determined by the overall product line strategy.

While the characteristics of the absorbent products contained within the package A1-A8 are the same, each package A1-A8 contains products that have a different combination of size and absorbency, relative to the other packages A1-A8. For ease of discussion, each package A1-A8, is said to have the same characteristics (e.g., size and absorbency) as the absorbent products contained within the package A1-A8. In one embodiment of the present invention, the product line 100 contains two or more packages A1-A8 that share the same size characteristic, while having different absorbency characteristics. For instance, in the product line 100 shown in FIG. 2, packages A2 and A5 have the same size, but package A5 has a higher absorbency than package A2. Similarly, packages A4 and A7 have the same size, but package A7 has a higher absorbency than package A5. It also may be possible for more than two packages A1-A8 within a product line 100 to have the same size, and different absorbencies. In another embodiment of the invention, the product line 100 contains two or more packages A1-A8 that share the same absorbency characteristic, while having different size characteristics. For example, in the product line shown in FIG. 2, packages A3 and A4 have the same absorbency, but package A4 has a larger size than package A3. Similarly, packages A5 and A6 have the same absorbency, but package A6 has a larger size than package A5. It also may be possible for more than two packages A1-A8 within a product line 100 to have the same size, and different absorbencies. Of course, a product line 100 of the present invention may contain two or more packages A1-A8 having the same size and different absorbencies; as well as two or more packages A1-A8 having the same absorbency and different sizes. The number and combination of packages A1-A8 within the product line 100 may be determined by the overall product line strategy.

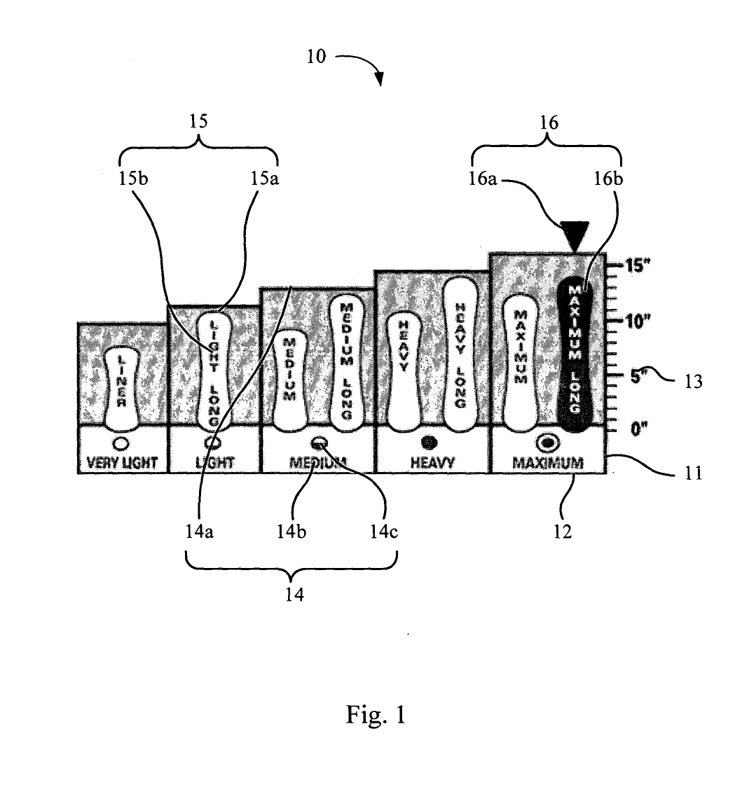

It is contemplated that the device of the present invention is particularly useful in identifying absorbent products of such a product line. FIG. 1 illustrates an embodiment of a device 10 of the present invention, where the device is a graphical representation having a first axis 11 extending vertically, and a second axis 12 extending horizontally. The first axis 11 generally corresponds to size designations 13, while the second axis 12 generally corresponds to absorbency designations 14. The device 10 comprises a plurality of absorbent product designations 15, with each absorbent product designation 15 being associated with a combination of one size designation and one absorbency designation.

The absorbent product designations 15 may comprise any information that identifies an absorbent product and each product designation 15 represents a particular product in a line of related products. The product designation may identify products by textual description, such as by number or letter (e.g., “1” or “A”), by direct description or a name (e.g., a descriptive term or a trademarked product name or brand name), or by indirect description or word association (e.g., “Light” or “Super”). The product designation also may identify products by color association, or by the use of graphics such as, for example, boxes, bars, partitions, graphical codes, graphical points, graphical plots, lines or symbols (e.g., the shape of the product). The product designation also may comprise a combination of any of the above product designation systems, or others not specifically listed herein, to identify a product.

In the embodiment shown in FIG. 1, the absorbent product designation 15 comprises two systems of identification: a graphical representation of the shape of the product 15a; and a textual description comprising the name of the product 15b. Each of the absorbent product designations is associated with one size designation 13 and with one absorbency designation 14, which identify the size and absorbency of the identified absorbent product, respectively. Although two absorbent product designations are used to identify the absorbent product, one of ordinary skill in the art will appreciate that either one of these product designations 15 may be sufficient alone, and that other designations or combinations of designations may be used instead.

It is not necessary that all of the product designations present on the device identify absorbent products that are concurrently being manufactured or offered for sale. For instance, it is possible that one or more product designations identify absorbent products that were at one time manufactured, and were thereafter discontinued. It is also possible that one or more product designations identify absorbent products that have never existed, and may or may not be intended to be manufactured or offered for sale. It is also possible that one or more product designations contain a place holder, such as a “coming soon” designation. It is also possible for the product designation to reference an absorbent product of a different product line from the same or different manufacturer.

The size designations directly or indirectly identify the size of the absorbent articles represented by the product designations 15. In one embodiment, the size designations may indicate the actual, approximate or relative physical dimensions of an absorbent article. For example, size designations can denote the actual physical measurements of length, width, thickness, volume or area of an absorbent article. Alternatively, the size designation may identify a size by a textual description, such as by numbers or letters (e.g., “Size 1” or “Size A”), by direct description (e.g., “Small” or “Large,” “Thick” or “Thin”), or by indirect description or word association (e.g., “Junior,” “Overnight”). The size designation also may identify a size by color association (e.g., use of one color to signify one size and another color to signify another size), or by the use of graphics such as, for example, boxes, bars, partitions, graphical codes, graphical points, graphical plots, lines or symbols (e.g., the use of one symbol to signify one size and another symbol to signify another size). The size designation also may comprise a combination of any of the above size designation systems to indicate the size of an absorbent article.

FIG. 1 shows an embodiment employing a combination of size designations. In FIG. 1, the size designation comprises a scaling index 13 along axis 11, representing a physical measurement of the length of an absorbent product, measured in inches. Each of the absorbent product designations 15 has a vertical dimension that corresponds to the length of the absorbent product, as measured by the scaling index 13 on axis 11. Thus each product designation 15 is graphically associated with a size designation that indicates the length of the absorbent product by reference to the scaling index 13. It will also be seen that FIG. 1 also provides a size designation for each absorbent product designation 15 by depicting each product designation 15 proportionally with its relative length. Thus, even if the scaling index 13 were not provided, a user would still be able to identify the size designations of each product designation by comparing their relative sizes. Of course, other size designations, including others not specifically described herein, also may be used in conjunction with, or in lieu of, those described herein with reference to FIG. 1.

As can be seen in FIG. 1, the association between the product designations 15, size designations and absorbency designations 14 indicates that numerous product designations 15 correspond to the same size designations. For example, the “Light Long” and “Heavy” product designations 15 both correspond to the same size designations, as measured by the scaling index 13. That is to say, these two product designations 15 are approximately the same size. Similarly, the “Medium Long” and “Maximum” product designations 15 are the same size, and so are the “Heavy Long” and “Maximum Long” product designations 15. Using the embodiment of FIG. 1, a user can readily distinguish between the various size products of a given product line, and identify those that are most suitable for the user's size requirements. By clearly indicating the size and absorbency characteristics for a range of similar products, this embodiment of the invention overcomes the conventional obstacle of helping users identify products that meet both their absorbency requirements and their fit requirements. As such, the users understand that they do not have to sacrifice a comfortable fit to obtain the desired absorbency level (or vice-versa).

The absorbency designations 14 include direct or indirect identification of the absorbency level of an absorbent article. The term “absorbency” refers to the absorbency-related properties of an absorbent article, such as the amount of fluid that an absorbent product will absorb or retain, under specified conditions (e.g., time, load, type of fluid). Examples of measured properties that can be used to characterize the absorbency of an absorbent article include, but are not limited to, strikethrough, rewet, overall capacity, and absorbency under load. In the present invention, the absorbency designation may indicate the actual physical measurements of the absorbency of an absorbent article, numerically or by relative designations. For example, absorbency designations can denote properties or values obtained during testing of strikethrough, rewet, overall capacity, absorbency under load testing, or any combination of the above tests. The absorbency designations also may identify absorbency by using a textual description, such as by a number or letter (e.g., “1” or “A”), by direct description (e.g., “Light” or “Heavy”), or by indirect description or word association (e.g., “Ultra” or “Super”). The absorbency designation also may identify absorbency by color association (e.g., use of one color to signify one absorbent level and another color to signify another absorbent level), or by the use of graphics such as, for example, boxes, bars, partitions, graphical codes, graphical points, graphical plots, lines or symbols (e.g., the use of one symbol to signify one absorbent level and another symbol to signify another absorbent level). Of course, the absorbency designation also may comprise a combination of any of the above absorbency designation systems, or other systems not specifically listed herein, to indicate the absorbency of an absorbent article.

In the embodiment of FIG. 1, the absorbency designation comprises three identification systems: a bar 14a having a length that graphically represents the absorbency of the absorbent products within the bar 14a, relative to the other absorbent products; a textual description 14b of the absorbency of the absorbent products; and a symbol 14c that identifies the absorbency of the absorbent products relative to one another. Each product designation 15 is associated with one or more of these identification systems, thereby identifying the absorbency level of the absorbent products represented by the product designations 15. Although the absorbency designations 14 are used in combination in FIG. 1, any one of these absorbency designations may be sufficient to identify the absorbency of the products, either alone or in combination with other absorbency designations. It should also be noted that, in this embodiment, the first axis 11 corresponds both to product size, by reference to the scaling index 13, and also to absorbency, by comparing the bar-graph-like bars 14a to one another.

The device of the present invention also may include multiple absorbent product designations 15 that refer to the same absorbency designations 14. For example, as shown in FIG. 1, the “Medium” and “Medium Long” product designations 15 are both associated with the same “Medium” absorbency designation 14. Stated differently, the “Medium” and “Medium Long” products have approximately the same absorbency. The same is true for the “Heavy” and “Heavy Long” products and the “Maximum” and “Maximum Long” products. As previously discussed with respect to the size designations, this association improves the users' ability to evaluate the absorbent properties of the various products represented by the product designations 15.

The embodiment shown in FIG. 1 also includes a content indicator 16 that is used to identify the particular product that is contained in the package upon which the device 10 is placed. The content indicator 16 may be any means suitable for marking one of the product designations 15 so that it stands out, and is differentiated from the rest of the product designations 15. The content indicator 16 may identify a product by textual description, such as by number or letter, by direct description, or by indirect description or word association. The content indicator also may identify a product by color association (e.g., shading or coloration), or by the use of graphics such as, for example, boxes, bars, partitions, graphical codes, graphical points, graphical plots, lines or symbols (e.g., an arrow or a circle). The content indicator is 16 also may comprise a punch-hole or may otherwise comprise a physical alteration or marking or the device. The content indicator 16 also may comprise a combination of any of the above content indicator systems, or others not specifically described herein, to identify a product. For example, as shown in FIG. 1, the content indicator 16 may comprise a combination of two systems: an arrow 16a pointing to the product designation corresponding to the product in the package, and a shading or coloration 16b of the product designation corresponding to the product in the package so that it stands out from the other product designations 15 on device 10.

The device 10 of the present invention, as shown in FIG. 1, is particularly suited as a method for marking a package of absorbent articles. In the embodiment of FIG. 1, for example, a package comprising the device 10 would contain “Maximum Long” absorbent products, as indicated by the product designation 15 in combination with the content indicator 16. The product designation 15 is associated with a size designation 13, and an absorbency designation 14, which characterize the absorbent product as having a “Maximum” absorbency level, and a size (length) of approximately 14 inches.

It is also anticipated that, the device 10 of the present invention also may be used on or as part of an in-store display where the displayed products are offered for sale, in print or electronic advertising, promotional materials, or in other contexts in which information is presented regarding absorbent products that are available as one of a series of products in a particular brand or product line. In such a case, the content indicator 16 may be omitted if the device 10 is used to identify the entire product line, rather than to specify a particular product.

While the invention has been described with reference to particularly preferred embodiments and examples, those skilled in the art will appreciate that various modifications may be made thereto without significantly departing from the spirit and scope thereof.

1. A visual identification device for absorbent products, the device comprising:
two or more size designations;
two or more absorbency designations;
two or more absorbent product designations;
an association between each of the two or more absorbent product designations and a combination of one of the size designations and one of the absorbency designations, wherein the association identifies one or more absorbent product designations that are predicted to satisfy a user's fit and absorbency requirements; and
wherein the association identifies at least two absorbent product designations that correspond to one of the two or more size designations.

2. The visual identification device according to claim 1, wherein the association identifies at least two absorbent product designations that correspond to one of the two or more absorbency designations.

3. The visual identification device according to claim 1, wherein at least one of the two or more absorbent product designations comprises a name.

4. The visual identification device according to claim 1, wherein at least one of the two or more absorbent product designations comprises a product shape.

5. The visual identification device according to claim 1, wherein each of the absorbent product designations identifies a respective bladder control product.

6. The visual identification device according to claim 1, wherein the device is selected from the group consisting of a chart, a graphical representation, a table and a list.

7. The visual identification device according to claim 1, wherein at least one of the size designations, the absorbency designations, and the absorbent product designations comprises a textual description.

8. The visual identification device according to claim 1, wherein at least one of the size designations, the absorbency designations, and the absorbent product designations comprises a graphical code.

9. The visual identification device according to claim 8, wherein the graphical code is selected from the group consisting of: words, letters, numbers, boxes, partitions, color codes, symbols, graphical points, bars, lines, and any combination thereof.

10. The visual identification device according to claim 1, wherein at least a portion of the visual identification device is displayed on a medium selected from the group consisting of: packaging materials, in-store display materials, advertising materials, promotional materials, and any combination thereof.

11. The visual identification device according to claim 1, wherein the visual identification device is displayed on a package for an absorbent article, the visual identification device further comprising:
a content indicator associating one of the two or more absorbent product designations with the absorbent article.

12. The visual identification device according to claim 1, further comprising a content indicator associated with one of the two or more absorbent product designations.

13. An absorbent product line comprising the visual identification device of claim 1.

14. A graphical representation for absorbent products, the graphical representation comprising:
a chart having a size axis and an absorbency axis, the size axis corresponding to size designations, and the absorbency axis corresponding to absorbency designations;
two or more absorbent product designations located on the chart; and
an association between each of the two or more absorbent product designations and a combination of one of the size designations and one of the absorbency designations; wherein the association identifies one or more absorbent product designations that are predicted to satisfy a user's fit and absorbency requirements; and
wherein the association identifies at least two absorbent product designations that correspond to one of the two or more size designations.

15. The graphical representation according to claim 14, wherein the association identifies at least two absorbent product designations that correspond to one of the two or more absorbency designations.

16. The graphical representation according to claim 14, wherein each of the absorbent product designations identifies a respective bladder control product.

17. The graphical representation according to claim 14, wherein at least one of the size designation, the absorbency designation, and the absorbent product designation comprises a textual description.

18. The graphical representation according to claim 14, wherein at least one of the size designation, the absorbency designation, and the absorbent product designation comprises a graphical code.

19. The graphical representation according to claim 18, wherein the graphical code is selected from the group consisting of: words, letters, numbers, boxes, partitions, color codes, symbols, graphical points, bars, lines, and any combination thereof.

20. The graphical representation according to claim 14, further comprising a content indicator associated with one of the two or more absorbent product designations.

21. An absorbent product line comprising the graphical representation of claim 14.

22. A method for marking an absorbent article package, the method comprising:
marking two or more absorbent product designations;
marking two or more absorbency designations, wherein each absorbent product designation is associated with one or more absorbency designations;
marking two or more size designations, wherein each absorbent product designation is associated with one or more size designations, and at least one size designation is associated with two or more absorbent product designations associated with different absorbency designations.

23. The method according to claim 22, wherein at least one of the two or more absorbent product designations comprises a name.

24. The method according to claim 22, wherein at least one of the two or more absorbent product designations comprises a product shape.

25. The method according to claim 22, wherein each of the two or more absorbent product designations identifies a respective bladder control product.

26. The method according to claim 22, wherein at least one of the size designations, the absorbency designations, and the absorbent product designations comprises a textual description.

27. The method according to claim 22, wherein at least one of the size designations, the absorbency designations, and the absorbent product designations comprises a graphical code.

28. The method according to claim 27, wherein the graphical code is selected from the group consisting of: words, letters, numbers, boxes, partitions, color codes, symbols, graphical points, bars, lines, and any combination thereof.

29. The method according to claim 22, further comprising:
marking a content indicator associated with one of the two or more absorbent product designations.

30. A package for containing absorbent products comprising:
two or more size designations;
two or more absorbency designations;
two or more absorbent product designations;
an association between each of the two or more absorbent product designations and a combination of one of the size designations and one of the absorbency designations, wherein the association identifies one or more absorbent product designations that are predicted to satisfy a user's fit and absorbency requirements; and
wherein the association identifies at least two absorbent product designations that correspond to one of the two or more size designations.

31. The package according to claim 30, further comprising a content indicator that associates one of the absorbent product designations with an absorbent product that is intended to be contained within the package.

32. The package according to claim 30, wherein one of the two or more absorbent product designations comprises a name.

33. The package according to claim 30, wherein one of the two or more `absorbent product designations comprises a product shape.

34. The package according to claim 30, wherein each of the absorbent product designations identifies a respective bladder control pad.

35. The package according to claim 30, wherein the device is selected from the group consisting of: a chart, a graphical representation, a table, a list, and any combination thereof.

36. The package according to claim 30, wherein at least one of the size designation, the absorbency designation, and the absorbent product designation comprises a textual description.

37. The package according to claim 30, wherein at least one of the size designation, the absorbency designation, and the absorbent product designation comprises a graphical code.

38. The package according to claim 37, wherein the graphical code is selected from the group consisting of words, letters, numbers, boxes, partitions, color codes, symbols, graphical points, bars, lines, or a combination thereof.

39. An absorbent product line comprising the package of claim 30.

40. An absorbent product line comprising:
two or more absorbent products, whereby each of the two or more absorbent products is characterized by a combination of an absorbency designation and a size designation, and
each of the two or more absorbent products are characterized by a different combination of absorbency designation and size designation, and
at least two of the two or more absorbent products are characterized by the same absorbency designation and different size designations.

41. The absorbent product line according to claim 40, wherein at least two of the two or more absorbent products are characterized by the same size designation and different absorbency designations.

42. An absorbent product line comprising:
two or more absorbent products, whereby each of the two or more absorbent products is characterized by a combination of an absorbency designation and a size designation; and
each of the two or more absorbent products are characterized by a different combination of absorbency designation and size designation; and
at least two of the two or more absorbent products are characterized by the same size designation and different absorbency designations.

43. An absorbent product line comprising:
a plurality of packages; and
a plurality of products having a similar construction, each of the products having a size designation and an absorbency designation;
wherein each of the packages contains products having the same size designation and the same absorbency designation; and
wherein each of the packages has the same size designation and absorbency designation as the products it contains; and
wherein at least two of the packages in the product line have the same size designation and different absorbency designations.

44. An absorbent product line according to claim 43, wherein at least two of the packages in the product line have the same absorbency designation and different size designations.

45. An absorbent product line comprising:
a plurality of packages; and
a plurality of products having a similar construction, each of the products having a size designation and an absorbency designation;
wherein each of the packages contains products having the same size designation and the same absorbency designation; and
wherein each of the packages has the same size designation and absorbency designation as the products it contains; and
wherein at least two of the packages in the product line have the same absorbency designation and different size designations.
